(19) United States
(12) Patent Application Publication
(10) Pub. No.: US 2025/0182170 A1
(43) Pub. Date: Jun. 5, 2025

(54) Provider-Centric Fee Analysis, Multi-Staged Pricing Modification, and Practice Management Customization

(71) Applicant: eAssist, Inc., American Fork, UT (US)

(72) Inventors: James Victor Anderson, American Fork, UT (US); James C. DiMarino, Naples, FL (US); Greg Nelson Grobmyer, Signal Mountain, TN (US); Tiffany Sue Wesley, Overland Park, KS (US); Amara Cashion, Spotsylvania, VA (US); Mason Blair, Matthews, NC (US); Julie Devinney, Mount Holly, NC (US); Amanda Hoyle, Mount Holly, NC (US); Denise Connett, Belmont, NC (US)

(21) Appl. No.: 18/936,995

(22) Filed: Nov. 4, 2024

Related U.S. Application Data
(60) Provisional application No. 63/595,885, filed on Nov. 3, 2023.

(51) Int. Cl. G06Q 30/04; G16H 15/00

(52) U.S. Cl. CPC G06Q 30/04; G16H 15/00

(57) ABSTRACT

A dental practice management system and method for modifying procedure fee structures. The system may include automated modules for data retrieval, normalization, and analysis. The system standardizes fee data from diverse sources, compares practice fees against percentile-based targets, and generates incremental adjustment recommendations, such as phased increases over multiple months, to align with industry benchmarks and/or percentile-based targets. The system offers practitioners a user-configurable interface for modifying target percentiles and staging plans. A report may identify deprecated codes and/or underutilized ones. The system facilitates staged fee increases to minimize patient dissatisfaction and maintain competitive positioning.

### PRIORITY APPLICATIONS

This application claims the benefit of and priority to U.S. Provisional Patent Application No. 63/595,885 filed on Nov. 3, 2023, titled “Practice Analysis and Reporting System with Multi-Staged Fee Increase Recommendations,” which is hereby incorporated by reference in its entirety.

### TECHNICAL FIELD

This disclosure relates to medical and dental billing, documentation, and records.

### BRIEF DESCRIPTION OF THE DRAWINGS

FIG. 1 illustrates a co-pilot portal computing system, according to one embodiment.

FIG. 2 illustrates a block diagram of a co-pilot portal computing system interacting with a dental office practice management system, according to one embodiment.

FIGS. 3A-C illustrate block diagrams of various examples of a co-pilot portal system interacting with a dental office practice management system and a medical imaging analysis system, according to one embodiment.

FIG. 4 illustrates a block diagram of a claim scrubber system with a conditions-based rules engine processor, according to one embodiment.

FIG. 5 illustrates a block diagram of a machine-learning claim scrubber system of a co-pilot portal system, according to one embodiment.

FIG. 6A illustrates a subsystem configured to validate a claim based on an analysis of a medical image using a machine learning model, according to one embodiment.

FIG. 6B illustrates a subsystem configured to generate or augment the descriptive narrative of a claim based on an analysis of a medical image using a machine learning model, according to one embodiment.

FIG. 7 illustrates a block diagram of a narrative generation subsystem used to generate a claim narrative prior to processing via the machine-learning claim scrubber system, according to one embodiment.

FIG. 8 illustrates a flow chart of an example method to automatically generate or augment a descriptive narrative for a dental billing code in a claim using machine-learning-based image analysis of an X-ray, according to one embodiment.

FIG. 9 illustrates an image showcasing the capabilities of artificial intelligence to identify pathologies in X-ray images, according to one embodiment.

FIG. 10 illustrates a list of dental codes that might be entered into a claim in connection with work performed on a patient whose X-rays correspond to those illustrated in FIG. 9, according to one embodiment.

FIG. 11 illustrates a block diagram of a fee practice analysis computing system, according to one embodiment.

FIG. 12 illustrates a block diagram of an example cost analysis and pricing recommendation system, according to one embodiment.

FIG. 13A illustrates an example of a first portion of a fee practice report with staggered or staged fee increase recommendations, according to one embodiment.

FIG. 13B illustrates another example of a first portion of a fee practice report with staggered or staged fee increase recommendations, according to one embodiment.

FIG. 13C illustrates an example fee report of an immediate fee increase plan, according to one embodiment.

FIG. 13D illustrates an example fee report for future fee increases by dental billing code, according to one embodiment.

FIG. 14 illustrates an example utilization report for an example dental code with a projected increase in revenue, according to one embodiment.

### DETAILED DESCRIPTION

There are several fields in which the various embodiments described herein can be utilized, including but not limited to medical, dental, veterinary, insurance, legal, etc. Aspects of the systems and methods described herein include technical solutions and implementations of hardware devices that differ from existing server-client interactions and human-machine interface interactions.

This disclosure provides a specialized system and method for analyzing, updating, and structuring dental procedure fees, using advanced data integration, error-checking, and predictive modeling tailored to meet the needs of dental practices managing insurance claims and fee schedules. The same systems and methods can be adapted for use in fields other than dentistry (e.g., legal, medical, and other professional service industries). In various embodiments, the system retrieves and normalizes fee data from multiple independent sources based on geographic location (e.g., aggregated and/or normalized external source data), allowing dental practices to evaluate their fees relative to percentile-based target fees. Through this comparative analysis, the system generates recommendations for fee adjustments to align with target percentiles. The recommendations are, in many examples, based on local standards, user-specified goals or limitations, and industry trends. The system automates multi-source data integration and leverages predictive analysis on patient feedback and insurance acceptance rates. The system enables dental practices to improve their revenue streams and billing compliance more effectively than traditional, manual approaches, and without human bias.

Many embodiments of the system described herein incorporate an error detection module that validates imported data, identifies inconsistencies, and prompts users to correct or complete missing data. The error detection module ensures the accuracy of fee data, which can otherwise suffer from manual entry errors, especially given the large volume of procedure codes and associated fees involved. Unlike generic data-entry automation, this error-checking process includes a manual user-interactive feedback mechanism as a technical solution to a significant bottleneck in dental billing processes, where incomplete or inaccurate data could lead to compliance risks or revenue losses. The error detection module, therefore, not only improves efficiency but also directly addresses accuracy challenges unique to the dental industry.

Various embodiments of the systems described herein include a predictive model that adapts over time based on historical data from patient feedback and/or insurance rejection/acceptance feedback. The system may adjust recommendations based on analyzed trends for underutilized and/or under-coded procedures. The system's predictive functionality provides a customized and evolving solution that traditional static systems lack. For example, when the system identifies that certain procedures are consistently underutilized, such as the treatment of gingivitis based on demographic data, the system may recommend a change in billing practices and associated treatments to improve revenue capture. This continuous, feedback-based adaptation to data entry and dental fee analysis is more than the mere automation of dental code data entry. The system improves coding accuracy and procedural recommendations with real-time learning.

In various embodiments, the system may also include a module for dynamic adjustment of target percentiles, staging of fee adjustments over time, and percentile-based goal setting for revenue optimization. By offering practitioners control over these variables through an interactive reporting user interface, the system provides a practical application. Moreover, the system allows for a nuanced, data-driven approach to fee setting. For instance, a dentist or other user may set global percentile goals or may choose fee increases in staged increments. These practices can facilitate the management of revenue goals and patient satisfaction without resorting to static or arbitrary fee structures. The dental fee analysis and reporting system provides a dynamic interface, combined with real-time data processing, and is more than mere data automation and comparison.

The system described herein goes beyond static fee adjustment by allowing dental practices to customize fee changes dynamically. The user interface provides real-time visualization of potential financial impacts as adjustments are made. The graphical user interface provides a report that enables practitioners to explore different fee scenarios and observe projected revenue changes instantaneously, fostering informed decision-making. This real-time feedback loop supports user-driven experimentation with different strategies for fee adjustments, aligning with strategic practice goals. By enabling on-the-fly changes, this feature helps dental offices tailor fee updates in response to evolving market conditions or internal policies without waiting for post-process evaluations.

In various embodiments, the system identifies outdated, custom, or deprecated codes and replaces them with standardized codes, ensuring accuracy in record-keeping and legal compliance. The system may also recommend corrections or changes based on historical claim data relating to insurance carrier responses, rejections, and acceptances for specific coding and narrative combinations. The system improves the chances of receiving reimbursement.

In some embodiments, the system also forecasts revenue increases when complex fee adjustments arise due to the existence of multiple patient payer types (e.g., cash, Medicaid, PPO patients, etc.). The systems and methods described herein utilize multi-source fee data integration, predictive adjustments, error-checking, and dynamic percentile adjustments to directly address practical challenges in dental billing. These functionalities provide improvements in accuracy, efficiency, and compliance that are specific to the dental industry and would be difficult, if not impossible, to replicate through conventional manual or automated systems. For example, continuous feedback-driven adaptations, automatically updated graphical user interfaces, and real-time processing are innovative technical solutions that improve revenue optimization and procedural compliance.

Documenting dental healthcare, as required when submitting dental insurance claims, requires accurate documentation of procedures performed. Current Dental Terminology (CDT) codes are a standardized and frequently revised set of dental codes used to achieve uniformity, consistency, and specificity when documenting dental treatments. Similarly, international classification of diseases (ICD) codes and current procedural terminology (CPT) codes may also be used in various dental and medical documentation and claims processing. For example, ICD-10 codes may be used in claims filed for dental benefits to inform the payor why the procedure was performed and the associated disease, illness, symptom, or disorder. The ICD-10 code categories K00 to K95 describe diseases of the digestive system and include diseases of the mouth. Throughout this application, usage of the terms “CDT dental codes” or “dental codes” should be understood to encompass other standardized coding schemes that may be useful for medical or dental billing in various circumstances. Dental offices may refer to a code as an ADA code in some instances with reference to the American Dental Association (ADA) and/or simply as a “dental code.”

Dental offices frequently charge a fixed or standardized fee for procedures associated with each specific dental code. For example, a dental office may use dental code D0230 for an intraoral-periapical EA ADDL Radiographic Image. The dental office may charge a standard fee of $25 for each image. Determining the price to charge for each dental code can be difficult—especially when insurance reimbursements are often much lower than the stated or standardized fee.

The presently described systems and methods provide tools for a dental office to obtain a fee analysis and report with recommendations for updating billing dental codes, deleting expired or obsolete dental codes, increasing fees, increasing fees in stages, and the like. In some embodiments, the analysis and report may provide an analysis of how their usage of specific fee codes (procedures performed) compares to other practices. In various embodiments, the reports and recommendations are based on zip codes and other local practice information. In some embodiments, the system utilizes more than one database of average fees to ensure accuracy and justify the recommendations provided.

In some embodiments, the system tracks the fee increases made by each dental practice and records or receives feedback provided by patients, insurance companies, and the dental office regarding the acceptance of the fee increases. The system may utilize artificial intelligence to identify the sentiment of the feedback associated with each fee increase by dental code. The system may identify negative feedback associated with specific fee code increases, neutral or no feedback associated with some fee code increases, and positive feedback associated with some fee code increases. In some embodiments, the system may cease recommending fee increases for dental billing codes associated with negative feedback in excess of a threshold value.

In other embodiments, the system may stagger fee increases associated with dental billing codes for which previous fee increases resulted in negative feedback. The system may stagger the fee increases to be smaller and/or occur over a longer period of time. For example, instead of raising the fee for a particular dental code by 20%, the fee may be raised 5% immediately and then an additional 5% every six months until the target fee level, which may be based on a percentile-based target, is achieved.

The system may utilize a target fee level that is a percentage of the average fee charged for each dental code for a given zip code or other geographic area. The system may utilize a target fee level that is a percentage of the highest fee charged for each dental code for a given zip code. Alternatively, the system may utilize a target fee level for each dental code selected to place the dental fee charged by the dental office within a target percentile category. For example, the system may recommend fee increases to ensure that the dental office's fees place them among the highest 75th percentile, 85th percentile category, or 95th percentile of dental offices within their zip code or other geographic region.

Accordingly, the system may generate a staged fee increase plan for each procedure code, ensuring that no single fee increase exceeds the specified maximum percentage, adjustments are distributed across the specified number of fee adjustment stages, and procedure codes already at or above the percentile target are adjusted by not more than a predefined percentage threshold. For example, procedure codes already at or above the percentile target may be adjusted by not more than a predefined percentage threshold that is zero, a small percentage number, indexed to inflation, etc. The report and plan for staged price increases may include a timeline for each stage of price increases and the associated revenue for each stage.

This disclosure also includes a discussion of the use of artificial intelligence to facilitate, generate, and/or review insurance claim submissions. Any of the various aspects of the presently described systems and methods can be combined or utilized separately from one another. Insurance claim forms frequently require a dentist (or associated personnel in a dental office) to identify the patient, date of service, CDT dental codes (or other codes) for the work performed, a narrative description that details and/or justifies the work performed in connection with each CDT dental code, the tooth number associated with each CDT dental code, and various other information as understood by those of skill in the art.

While the CDT dental code may specify many aspects of the treatment performed, the narrative description provides additional details relating to the work performed and/or justification for the work performed. Manual entry of narrative descriptions can be time-consuming and susceptible to human error. There is a temptation to re-use (e.g., copy and paste) the same generic narrative description in connection with a given CDT dental code. However, insurance claim submissions with generic narrative descriptions are more likely to be rejected by insurance carriers than customized and highly detailed narrative descriptions.

Various artificial intelligence (AI) algorithms, including trained machine learning models, such as those utilizing neural networks (e.g., convolutional neural networks), have been demonstrated that can read dental X-rays and identify areas of concern, such as decay, bone loss, cavities, abscesses, enamel levels, dentin levels, pulpal involvement, or other pathologies and characterizations. However, these tools have generally been proposed to replace or assist dental practitioners in identifying the pathologies, confirming that no pathologies have been missed, and/or confirming the findings of the dental practitioner. In these instances, the AI-based systems receive X-rays and process the images to find and identify all possible pathologies with varying degrees of accuracy and specificity.

The presently described systems and methods propose using AI-based X-ray analysis not to diagnose pathologies in the first instance but rather to assist in the automated, customized drafting of a narrative description, chart notes, referral notes/forms, etc. to accompany each CDT dental code in dental documentation, such as in insurance claim forms. In some instances, the insurance claim forms limit the number of characters or words that can be submitted. In such cases, the system may utilize known abbreviations and shortcut terminology to accurately describe, for each dental code, the pathology that existed prior to the dental procedure and the nature of the dental work performed. In some instances, the narrative generated by the system may be based on an existing narrative provided by the user, notes available in the practice management system of the dental office regarding the work performed, and/or notes stored in conjunction with the X-rays.

Various aspects of the systems and methods outlined above are described in the context of a co-pilot portal CPP computing system. Many features of the co-pilot portal computing system described herein provide additional context for an improved understanding. It is appreciated that an AI-assisted narrative generation system may be implemented as a stand-alone system or as one subsystem within a broader suite of subsystems and functionalities.

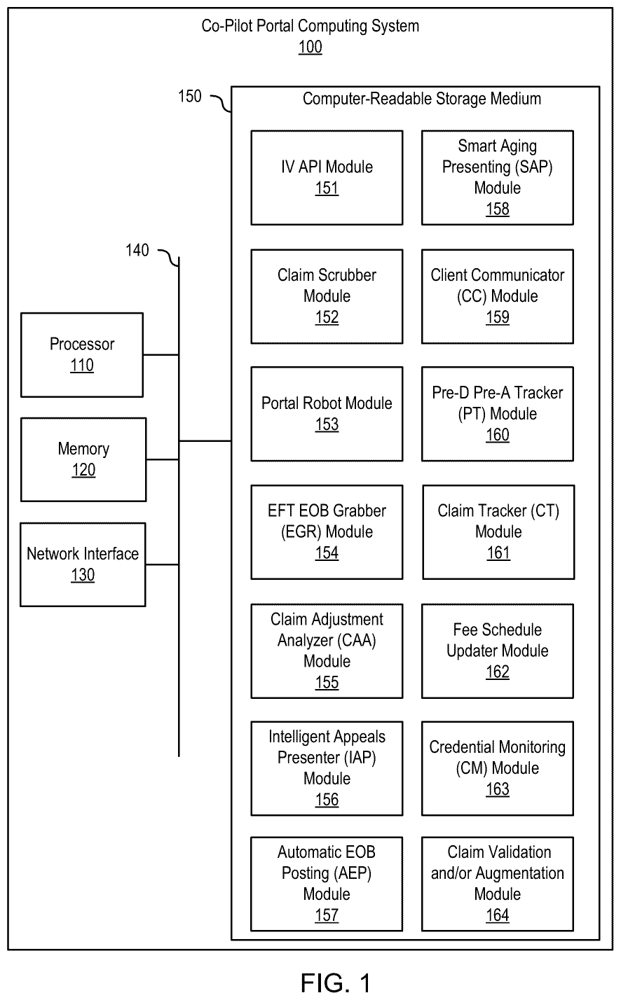

FIG. 1 illustrates a co-pilot portal computing system 100, according to one embodiment. As illustrated, each of the functionalities described herein may be implemented as instructions stored on a non-transitory computer-readable medium 150 that, when executed by the processor 110 in conjunction with the memory 120 and network interface 130 connected by the bus 140, cause the co-pilot portal computing system 100 to implement the various functionalities described herein. In various embodiments, the modules 151-164 may additionally include or be alternatively implemented as application-specific integrated circuits (ASICs), via programmable logic devices, and/or other hardware computing components.

The co-pilot portal computing system 100 provides numerous benefits to dental office personnel, dental office owners, dental office management platforms, dental office service providers, and the like. For example, the co-pilot portal computing system 100 enables a practice to work more efficiently by utilizing machine learning to implement learned “best practices.” The co-pilot portal computing system 100 may allow less-skilled employees to manage some of the dental office functions and/or reduce their learning curve so that they can be more efficient. For example, the co-pilot portal computing system 100 may allow employees to accomplish many of the tasks that previously required a highly skilled employee who is familiar/comfortable with claims and calling insurance carriers/patients. Thus, the co-pilot portal computing system 100 allows an employee to accomplish the tasks and perform the functions of an employee with years of training, skills, and/or experience.

The specific functionalities are described in greater detail below in conjunction with the associated bottlenecks and problems to be solved. However, in the context of the presently described architecture, the computer-readable storage medium 150 includes an IV API module 151. A claim scrubber module 152, a portal robot module 153, an EFT EOB grabber (EGR) module 154, a claim adjustment analyzer module 155, an intelligent appeals presenter module 156, an automatic EOB posting module 157, a smart aging presenting module 158, a client communicator module 159, a pre-D pre-A tracker module 160, a claim tracker module 161, a fee schedule updater module 162, a credential monitoring module 163, a claim validation and/or augmentation module 164, and/or any combination or permutations of the aforementioned modules or subsets of the aforementioned modules. Additional details regarding the functionalities and variations of the modules 151-164 of the co-pilot computing system 100 are described in U.S. patent application Ser. No. 17/937,192 filed on Sep. 30, 2022, titled “Centralized Practice Portal With Machine Learning Claim Processing,” which is hereby incorporated by reference in its entirety.

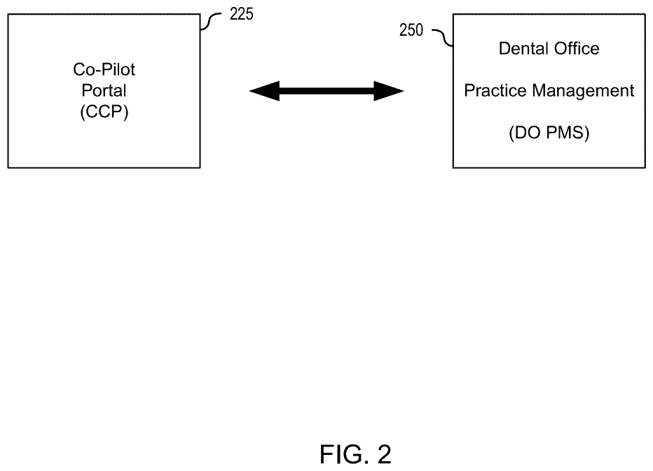

FIG. 2 illustrates a block diagram of co-pilot portal computing system 225 interacting with a dental office practice management system 250, according to one embodiment.

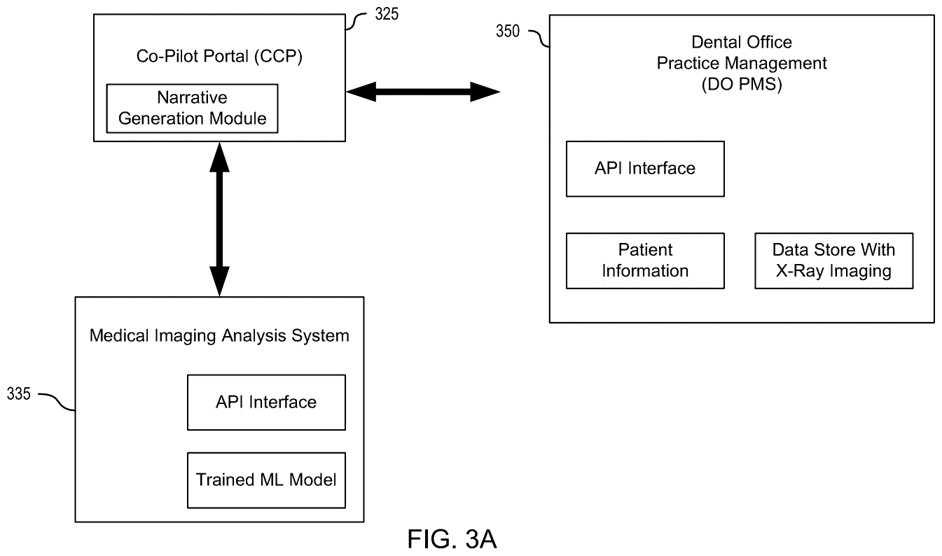

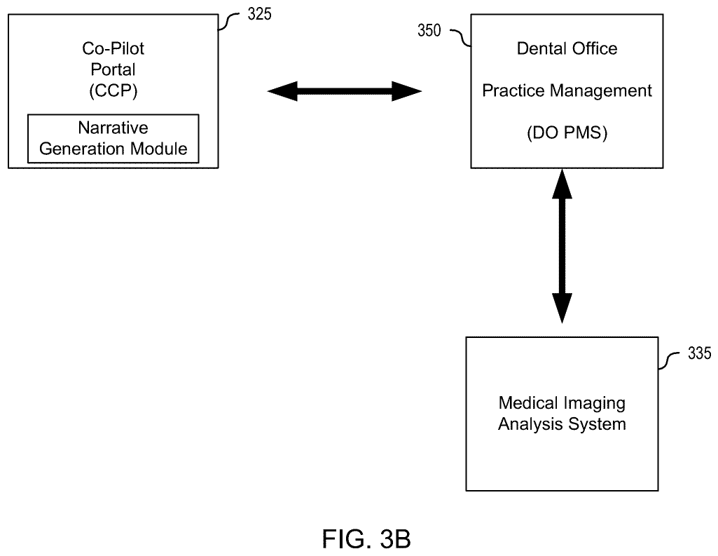

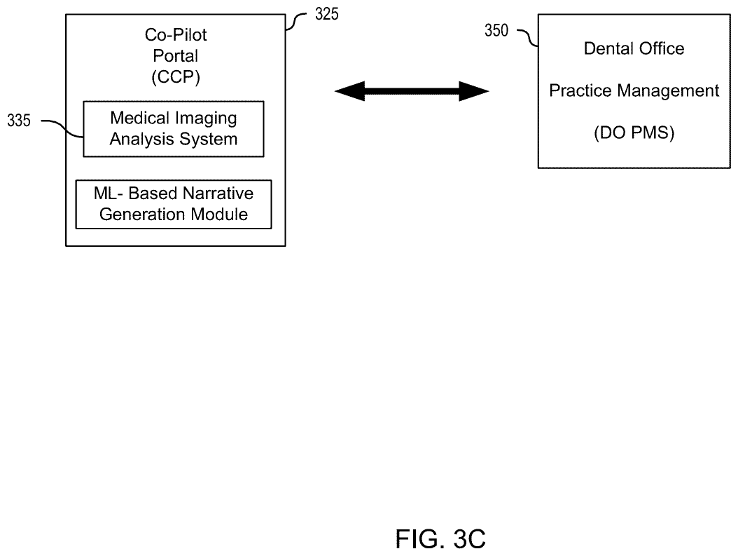

FIGS. 3A-C illustrate block diagrams of various examples of a co-pilot portal system interacting with a dental office practice management system and a medical imaging analysis system, according to one embodiment.

FIG. 3A illustrates a co-pilot portal computing system 325 that interacts with a dental office practice management system 350. The dental office practice management system 350 includes an API interface, a patient information database, and a database of X-ray images. The co-pilot portal computing system 325 also interacts with a stand-alone or external (e.g., third-party) medical imaging analysis system 335. The medical imaging analysis system 335 includes an API interface and a trained machine-learning (ML) model. According to various embodiments, the medical imaging analysis system 335 is an AI-based system that utilizes one or more artificial intelligence algorithms to detect pathologies in, characterize, annotate, and/or otherwise process dental X-ray images. The co-pilot portal computing system 325 includes a narrative generation module, such as a machine-learning-based narrative generation module, to generate a text-based description of the processed X-ray images using abbreviations and parlance common in the industry. The co-pilot portal computing system 325 may additionally or alternatively generate chart notes, referral notes/forms, etc.

FIG. 3B differs from FIG. 3A only in that the medical imaging analysis system 335 is connected to the dental office practice management system 350 instead of the co-pilot portal computing system 325. The co-pilot portal computing system 325 may request X-ray images from the dental office practice management system 350 via an API, a portal, or other customized integration. The co-pilot portal computing system 325 uses the AI-based medical imaging analysis system 335 to process X-ray images and returns the processed X-ray images with computer-readable pathology information (e.g., annotated images, marked-up comments or overlays, and/or various metadata). A natural language generation machine learning model to generate a narrative (or chart notes, referral notes/forms, etc.) for a specific dental code in a dental claim based on the processed X-ray images.

FIG. 3C differs from FIGS. 3A and 3B in that the medical imaging analysis system 335 is integrated as a subsystem within the co-pilot portal computing system 325. In this embodiment, the co-pilot portal computing system 325 may receive a partially completed or poorly completed dental claim that identifies a patient and includes at least one dental code. In some embodiments, the partially or poorly completed dental claim may also identify the tooth number associated with each dental code. The co-pilot portal computing system 325 may retrieve X-rays from the dental office practice management system 350 for the patient.

In some embodiments, the medical imaging analysis system 335 identifies which X-ray or X-rays are relevant to the dental code in the dental claim and identifies the tooth number, which can then be added to the claim. In other embodiments, the medical imaging analysis system 335 identifies the X-ray or X-rays that are associated with the pre-specified tooth number.

In some embodiments, a database associates each combination of dental code and tooth number with (i) a specific X-ray angle or X-ray type and (ii) a basic narrative. The medical imaging analysis system 335 uses this information to retrieve the appropriate X-ray. The medical imaging analysis system 335 uses one or more AI-based image analysis algorithms to augment the basic narrative with details specific to the analyzed X-ray. In various embodiments, an AI-based image analysis algorithm includes a machine learning model specifically trained to distinguish between teeth in an X-ray, distinguish between parts of a tooth, identify gums, identify bone, identify tooth enamel, identify decay, identify the pulp within a tooth, identify dentin, identify caries, identify various pathologies, and/or characterize spatial and relative regions, such as gross, occlusal, incisal, distal, facial, lingual, incipient, palatal, recurrent, mesial, etc.

The co-pilot portal computing system 325 uses the image analysis provided by the co-pilot portal computing system 325 to identify the specific pathology in the X-ray image that is associated with the dental code in the dental claim. The co-pilot portal computing system 325 then uses a machine learning algorithm within the narrative generation module to generate a narrative (or chart notes, referral notes/forms, etc.) that describes the pathology associated with the dental code and the dental work performed (based on the dental code and the identified pathology). The co-pilot portal computing system 325 may, in some embodiments, use the medical imaging analysis system 335 to analyze post-procedure X-rays to generate a narrative that describes the work performed as ascertainable from the post-procedure X-rays.

In the presently described embodiments, the medical imaging analysis system 335 may not perform a complete analysis of the X-ray image. Instead, the medical imaging analysis system 335 may be directed to analyze only the specific tooth (based on the provided tooth number) in the X-ray image and with the limited purpose of identifying the pathologies associated with the specific dental code. As such, the analysis may be faster, consume fewer resources, and/or more accurately identify the relevant information.

As an example, the co-pilot portal computing system 325 may process a claim for a patient named “Tim” that includes a dental code D2392 for tooth number 18. The co-pilot portal computing system 325 may retrieve Tim's X-rays from the dental office practice management system 350. The medical imaging analysis system 335 is used to process the retrieved X-rays using one or more AI algorithms to identify an X-ray that includes tooth number 18 and showcases the pathology associated with the dental procedures performed under dental code D2392. The medical imaging analysis system 335 may be used to determine if the decay has crossed the dento-enamel junction (DEJ) and, if so, the proximity of the decay to the pulp chamber and the probability that the tooth may require root canal treatment. The medical imaging analysis system 335 may be further used to verify that the bone levels indicate the tooth is worthy of repair. The co-pilot portal computing system 325 may utilize the ML-based narrative generation module to generate a customized narrative (or chart notes, referral notes/forms, etc.) that summarizes the identified characteristics and justifications for the dental work performed.

The system can also be used to offer treatment options and prognosis of success to cover the dentist legally and provide maximum options to the patient. Expanding on the example above, if the decay in tooth 18 has crossed the DEJ and has penetrated the pulpal chamber, the system can present options to the dentist to share with the patient including but not limited to: do nothing (always HAVE to offer the option to do nothing and the consequences) which has a high probability of allowing the decay to progress down the root, causing an abscess, pain, bone loss, swelling, fever, bacteremia, etc. Option two=RCT with prognosis based on bone levels, root curvature, etc. Option 3=extraction and the system would take into account the location of the tooth in the mouth to present replacement options: do nothing to replace it, don't replace since it is the last tooth in the mouth and has no antagonist, implant/flipper/bridge if it is in the aesthetic zone, etc. If the patient selects RCT for example, the medical imaging analysis system 335 may be further used to confirm that the bone levels of the tooth indicate a high probability of restorability based on bone support, depleted decay, and the amount of heart tissue above the gum line sorry bone level if there is a periapical pathology. The narrative can be customized to summarize the relevant information from most important and most severe to least important and least severe to stay within any character count limitations of the claim form. The co-pilot portal computing system 325 augments or modifies the narrative in the claim that is associated with the dental code to improve the chances that the claim is processed quickly and paid by the carrier.

Some dental codes may trigger specific analyses and narrative summaries to ensure claim processing and payment. For example, some CDT dental codes may trigger an analysis to cause the medical imaging analysis system 335 to identify and quantify the amount of bone loss resulting from a periodontal disease that justified the periodontal treatment associated with the specific code.

In some embodiments, the medical imaging analysis system 335 can operate differently than existing AI-based dental X-ray processing systems because the medical imaging analysis system 335 is told what to look for before the analysis starts. For example, an existing AI-based dental X-ray processing system may analyze an X-ray to identify all teeth, all pathologies, all bone loss, all decay, etc. The medical imaging analysis system 335 analyzes the tooth to identify the information relevant to a specific dental code. Thus, a general-purpose AI-based dental X-ray processing system might, for example, identify only a single spot of decay on a given tooth. However, the medical imaging analysis system 335 may be told to identify two areas of decay on the same tooth based on the dental code in the claim. The medical imaging analysis system 335 may identify the two areas that are most likely associated with the dental code, and the co-pilot portal computing system 325 may generate the appropriate customized narrative to fit the dental code-even when the general-purpose AI-imaging analysis system would not have characterized one of the areas as exhibiting sufficient decay to warrant a dental procedure.

The co-pilot portal computing system 325 extracts a dental code, tooth number or quadrant, surfaces, materials utilized, and/or other information in a dental claim to retrieve and process X-rays. The medical imaging analysis system 335 receives some or all the extracted information from the claim (e.g., the dental code, tooth number, quadrant, surfaces, materials utilized, etc.) to inform and guide the AI-based image analysis process. The output of the code-specific or guided AI-based image analysis process is used to generate a customized narrative detailing the characteristics of the tooth that justified the work performed under the specific dental code.

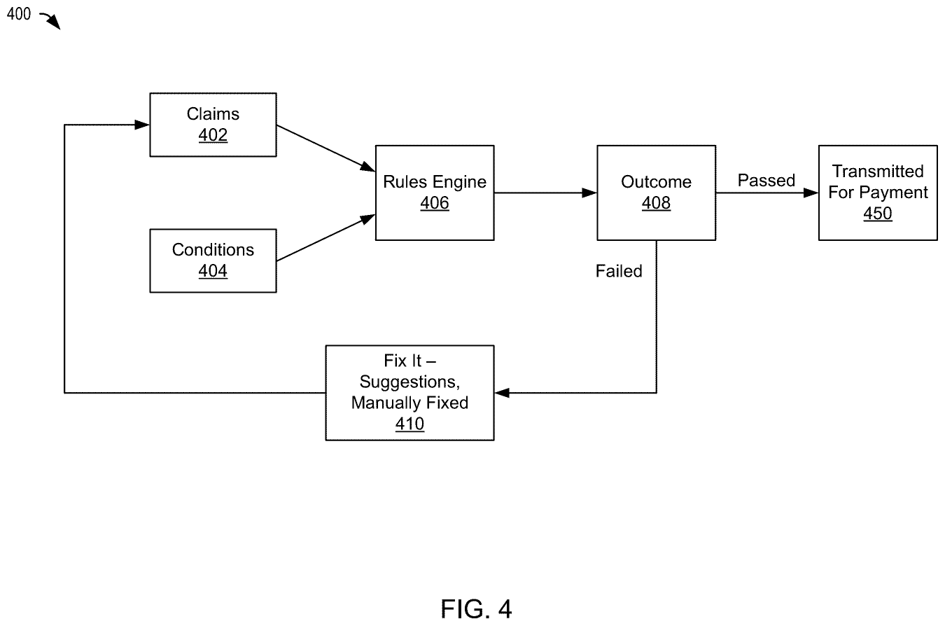

FIG. 4 illustrates a block diagram of a claim scrubber system 400 with a conditions-based rules engine processor, according to one embodiment. As illustrated, a rules engine 406 receives claims for submission to an insurance company (e.g., via a clearing house or via an insurance company dedicated insurance portal) along with conditions 404 that define the requirements for each claim for each insurance company (e.g., documentation, rules, codes, required information, etc.). Many of the conditions 404 may be specified by the insurance company, manually obtainable by phone call, accessible in printed literature, available via an API, or otherwise provided by the insurance company.

Other conditions 404 may be manually added by office personnel based on the collective knowledge of processing claims using legacy systems and approaches. For example, providers or dental office assistants may add manual conditions to limit submissions to certain days of the week, avoid holiday submissions, always include specific information that might not be required but results in faster processing in some instances, etc.

In some embodiments, the claim scrubber system 400 evaluates a claim at the time a claim is submitted for payment. The claim scrubber system 400 may determine that a claim is approved and complete the submission or determine that the claim is deficient or otherwise unlikely to be paid for another reason. As described herein, the claim scrubber system 400 may modify the claim and/or provide recommendations or suggestions for modifying the claim to increase the likelihood of payment.

In other embodiments, the claim scrubber system 400 is triggered and evaluates a claim at the time the claim is created. Evaluating the claim at the time of claim creation allows the original creator of the claim to evaluate any suggested modifications at the time of creation. In such an embodiment, all saved claims are ready for submission, having either been approved by the claim scrubber system 400, revised/updated by the claim creator, and/or manually approved by the claim creator despite any rejections or suggestions made by the claim scrubber system 400.

The rules engine 406 compares the claims with the conditions 404 and generates an expected outcome 408. The rules engine 406 may indicate a claim is likely to be paid and so has “passed” the analysis and can be transmitted for payment 450. Alternatively, the rules engine 406 may indicate a claim is deficient or otherwise likely to be unpaid or rejected. Failed claims may not be transmitted for payment 450 unless a human manually overrides the process and submits the claim for payment. In some embodiments, the claim scrubber system 400 identifies or suggests corrections, updates, documents, evidence, and/or other changes or modifications that could be made to the claim to improve the likelihood of payment. The claim scrubber system 400 may allow for the claim to be manually, or in some instances, automatically fixed, at 410, to become a new claim 402 to be re-analyzed and processed by the rules engine 406.

The claim scrubber system 400 can identify, based on particular carrier and dental billing codes, exactly what needs to be submitted (e.g., the conditions 404) for the claim to be paid. In some instances, the rules engine 406 can identify the information that is needed from within connected databases and automatically modify or update the claim. Some automatically updated/modified claims may be transmitted for payment 450 without any further review or human intervention. For example, if the claim was missing a patient's personal information, a date, a dental code was entered incompletely, or otherwise contained a factual error, the rules engine 406 may automatically update the claim using information in a connected database, the dental office practice management system, and/or practice notes relating to a particular procedure in the claim. In other instances, specific changes to a claim may require human verification and validation before being transmitted for payment 450.

ADA CDT codes are updated annually. As such, treatment proposed in CDT code format within a dental office software for a patient seen in December could no longer be relevant when the patient returns in January to have treatment completed. The system recognizes any ADA CDT code additions, deletions, and/or revisions and remaps the outdated code to the up-to-date and current ADA CDT code.

For example, the ADA CDT code to report Guided Tissue Regeneration-Resorbable Barrier (otherwise known as a resorbable membrane) in 2022 is D4266. The ADA's Code Maintenance Committee recognized the various common uses of guided tissue regeneration and approved three distinct code sets effective Jan. 1, 2023, one set for each of these uses. D6106 is specifically for GTR around an implant using a resorbable barrier. If a GTR-resorbable membrane is placed in conjunction with natural teeth (osseous surgery), report D4266. If a GTR-resorbable membrane is placed in conjunction with a bone socket graft, sinus lift, or edentulous area, report D7956. If a GTR-resorbable membrane is placed in conjunction with periradicular surgery, report D3432. The system identifies these code changes, and either identifies the claim for review by a human for correction/confirmation or replaces the ADA CDT code automatically based on information in the clinical chart notes.

With changes expected in the future to the ADA CDT code set and claim submission process, which would incorporate consistent and required usage of International Classification of Diseases (ICD) diagnosis codes and modifiers similar to that of the current medical claim submission process, the system also incorporates these changes either automatically or by way of a drop-down selection process to ensure accurate and timely dental claim submission. This change will impact dental offices in that most dental offices are unfamiliar with diagnosis codes or modifiers or how to apply them. Artificial intelligence and/or machine learning technologies track how these changes impact the claim submission process and which diagnosis codes and modifiers result in fewer claim denials, rejections, and appeals, as well as reimbursement rates for individual payors (insurance companies) so treatment plans can be presented accurately and patients can make informed healthcare and financial decisions.

As another example, a periodontal cleaning must have historical cleaning information attached to the claim to be paid. The rules engine 406 may determine that the claim for the periodontal cleaning does not include the required historical active treatment information, flag the claim, or prompt a user to provide the required information and update the claim. The rules engine can identify duplicate codes that are likely to be rejected. For example, an initial X-ray and an additional X-ray on the same day may require the use of different codes. If the initial claim used the same code, the rules engine 406 may flag the claims as being duplicative and suggest that either (1) one of the claims be deleted as an accidental duplicate or (2) that the ADA code for the second X-ray be updated to indicate that it was an additional X-ray.

As another example, the rules engine 406 may identify missing codes or errors based on a patient's age. Even if the patient's age is not in the claim being analyzed, the rules engine 406 can access the provider's PMS to determine the age of the patient and provide improved scrutiny and analysis. As an example, the system 400 may include conditions 404 specifying that “crown codes” are not payable for patients under the age of 4. The system may correct the age provided in the claim to match the age in the PMS system and/or identify the claim for review by a human for correction/confirmation. As another example, the conditions 404 may specify that anesthesia cannot be billed by itself due to insurance requirements and/or that it should not be billed by itself because it is indicative of a missing claim for the accompanying procedure. That is, even if the insurance company has no policy against billing anesthesia by itself, it is likely that another procedure was performed for which a claim should be submitted.

As another example, the rules engine 406 may evaluate a claim and determine that it satisfies the conditions 404 specified by insurance companies (e.g., insurance company conditions) but that additional conditions 404 for faster payment may be applicable (e.g., practice-specific conditions). For example, claims for X-rays and cleanings may be paid quickly, while claims for a crown may be paid more slowly. While the insurance company conditions 404 may allow for a combined claim that includes all three procedures, the practice-specific conditions may require or recommend that the claims be split into two (or three) different claims to facilitate the quickest possible payment for each claim. Thus, the system 400 may automatically split the claim into two or three different claims and submit them (assuming all other information is correct) and/or flag the claim (or updated/recommended claim) for review and approval before submission.

In various embodiments, the claim scrubber system 400 may manipulate the claim, patient, insurance, etc., while the PMS is down. In such instances, the claim scrubber system 400 tracks all changes in the order such changes were made. Once the PMS is working again, the claim scrubber system 400 updates the PMS to ensure that the claim scrubber system (and other aspects of the co-pilot portal system) are synchronized. The ordered changes are made to ensure that the two systems remain identical after the sync updates. In various embodiments, the system 400 may utilize a client-installed lightweight version of the open-source project Debezium to facilitate real-time and time-synchronous syncing between databases even when one of them is offline for a period of time.

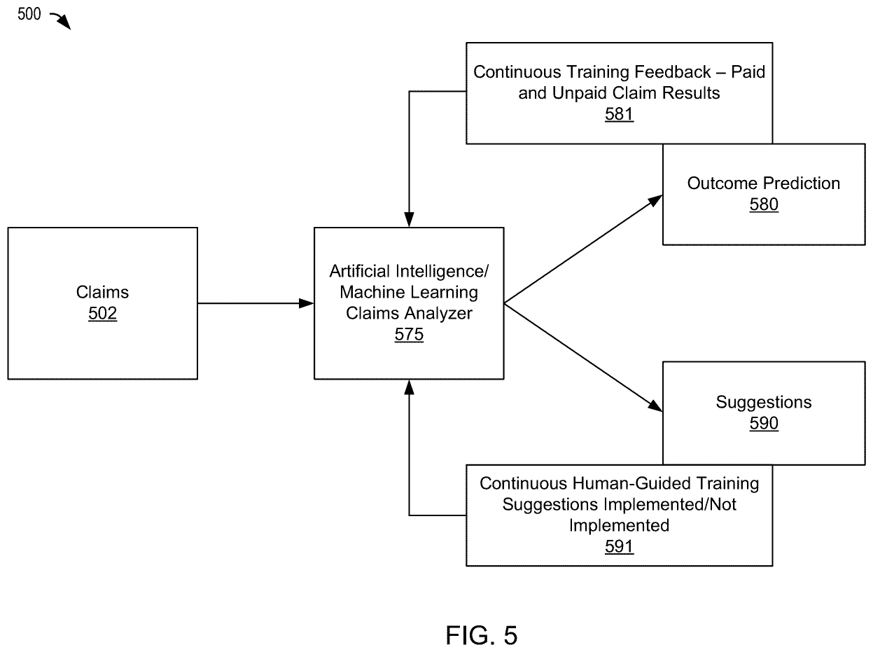

FIG. 5 illustrates a block diagram of a machine-learning claim scrubber system 500 of a co-pilot portal system, according to one embodiment. As illustrated, claims 502 are received by an artificial intelligence or machine learning claims analyzer 575 that is trained to generate an outcome prediction 580 and, in some instances, suggestions 590 for ways to improve the likelihood of claim payment. The outcome prediction may, for example, be provided in a pass or fail format or may indicate a percentage of likelihood that the claim will be processed and paid by the insurance company.

Claims that are associated with a positive outcome prediction 580 may be transmitted for payment without modification. The results of such submissions (i.e., the claims are paid or unpaid/rejected) are used as continuous training feedback 581 and fed back into the machine learning claims analyzer 575. In addition to the outcome accuracy feedback, the machine learning claims analyzer 575 may also receive human-guided training relative to the suggestions 590 that are made. That is, the machine learning model of the claims analyzer 575 may be trained based on suggestions 590 that are implemented by a human reviewer and suggestions 590 that are not implemented or marked as “bad suggestions.” Over time, the machine learning claims analyzer improves both the outcome prediction 580 and the suggestions 590 for improving the claim submission.

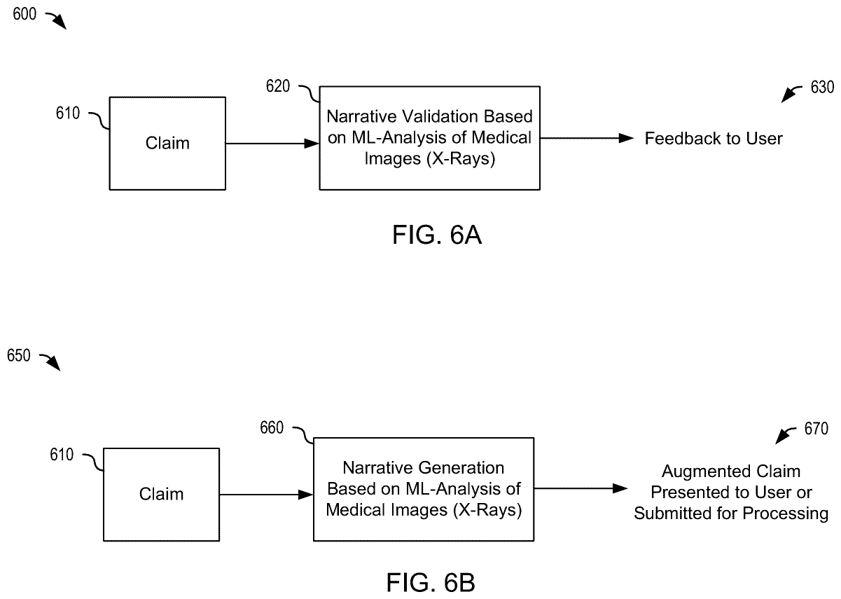

FIG. 6A illustrates a subsystem 600 configured to validate a claim 610 based on an analysis of a medical image using a machine learning model, according to one embodiment. In the illustrated example, a claim 610 is processed by a narrative validation module 620. The narrative validation module 620 uses an AI-based X-ray image analysis algorithm to analyze X-ray images to determine if the narrative associated with a particular dental code in the claim 610 is justified and accurate. An insurance carrier may utilize the illustrated system to analyze a claim in the context of X-rays provided by a dental practitioner as evidence to support a submitted claim. The feedback 630 may be in the form of a “pass or fail” output. Alternatively, the feedback 630 may be more detailed and recommend an alternative dental code that is better supported by the analyzed X-ray images, recommend a modified narrative, or identify specific concerns or errors within the narrative of the claim.

FIG. 6B illustrates a subsystem 650 configured to generate or augment the descriptive narrative of a claim 610 based on an analysis of a medical image using a machine learning model, according to one embodiment. As described in the context of FIGS. 3A-3C, the system may receive a claim 610 that includes a dental code without a narrative or with a narrative that needs to be augmented or improved. The system may include a narrative generation module 660 to generate a customized narrative for each dental code in the claim based on an analysis of X-ray images using an AI-based image analysis system. The augmented or newly added narrative 670 can be presented to the user for manual approval or automatically accepted to allow the claim to be directly submitted to the carrier.

In some embodiments, the subsystem 650 may be additionally or alternatively configured to validate, generate, and/or augment the descriptive narrative of a medical or dental claim based on an analysis of any of a wide variety of medical images, including but not limited to X-rays, periodontal charting, intro-oral images, scans, intraoral or wall-mounted images, panoramic images, ionizing imaging, non-ionizing imaging, computed tomography, cone beam computed tomography, magnetic resonance imaging, ultrasound images, cephalograms, etc.

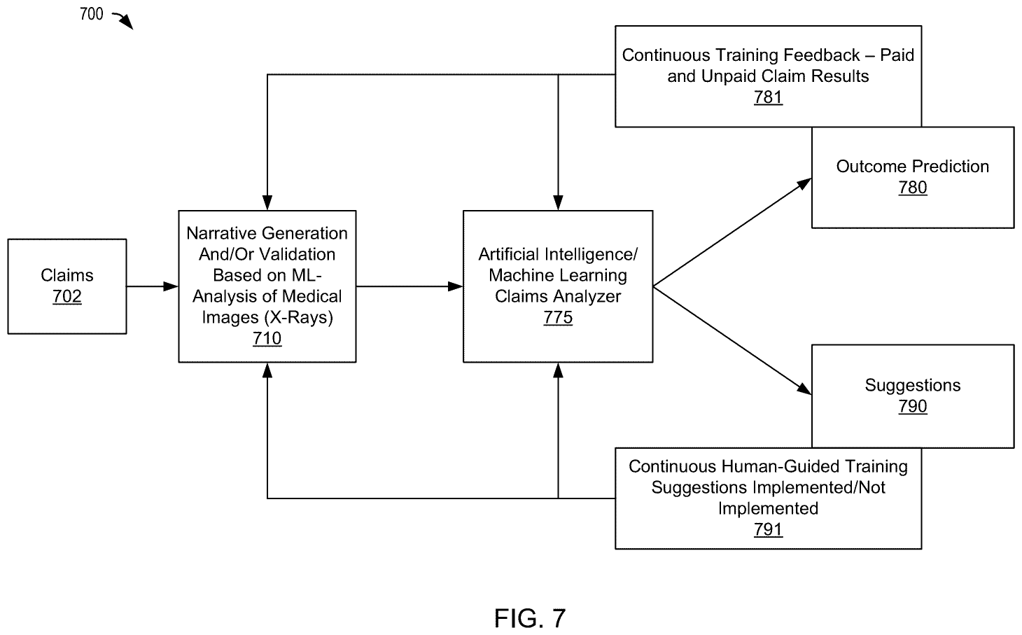

FIG. 7 illustrates a block diagram 700 of a narrative generation subsystem used to generate a narrative for each CDT code in a claim prior to processing the claim via the machine-learning claim scrubber system, according to one embodiment. As illustrated, claims 702 are received by the narrative generation and/or validation subsystem that utilizes machine learning or other AI-based medical image analysis. The system generates a customized narrative for each CDT code, at 710, in each of the claims 702 based on the AI-based X-ray analysis. The augment claims are fed into the machine learning claims analyzer 775, which is trained to generate an outcome prediction 780 and, in some instances, suggestions 790 for ways to improve the likelihood of claim payment. The outcome prediction may, for example, be provided in a pass or fail format or may indicate a percentage of likelihood that the claim will be processed and paid by the insurance company.

Claims that are associated with a positive outcome prediction 780 may be transmitted for payment without modification. The results of such submissions (i.e., the claims are paid or unpaid/rejected) are used as continuous training feedback 781 and fed back into the machine learning claims analyzer 775 and/or the AI-based X-ray image analyzer module to improve future narratives. In addition to the outcome accuracy feedback, the machine learning claims analyzer 775 may also receive human-guided training relative to the suggestions 790 that are made. That is, the machine learning model of the claims analyzer 775 and/or the AI-based X-ray image analyzer module may be trained based on suggestions 790 that are implemented by a human reviewer and suggestions 790 that are not implemented or marked as “bad suggestions.” Over time, the machine learning claims analyzer 775 and/or the AI-based X-ray image analyzer module improve.

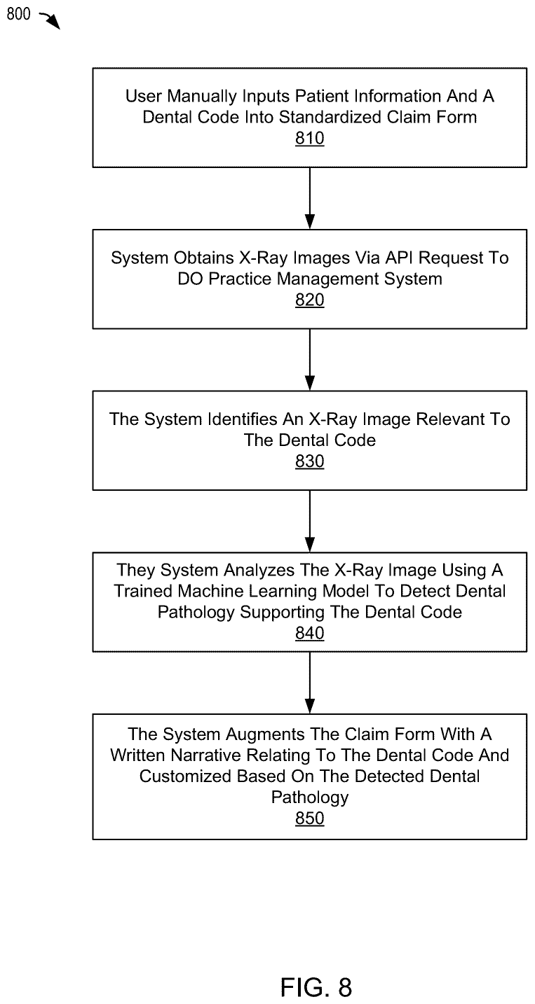

FIG. 8 illustrates a flow chart 800 of an example method to automatically generate or augment a descriptive narrative for a dental billing code in a claim using machine-learning-based image analysis of an X-ray, according to one embodiment. As illustrated, the process may include a user manually entering, at 810, patient information, a dental code, and an associated tooth number into a standardized claim form. The system may obtain, at 820, X-ray images that are relevant to the dental code and associated tooth number via an API request to the practice management system. In some embodiments, the system may review multiple X-rays to identify, at 830, the X-ray that is most relevant to the dental code and associated tooth number. The system may then analyze, at 840, the X-ray or X-rays using a trained machine-learning model or other AI-based medical image processing system to detect the pathology or other characteristics of the numbered tooth, surrounding teeth, bones, gums, or other information relevant to the dental code. The system then augments or generates, at 850, a narrative to be added to the claim for the specific dental code. The generated narrative is customized based on the specific information detected in the X-ray images by the AI-based medical imaging processing system.

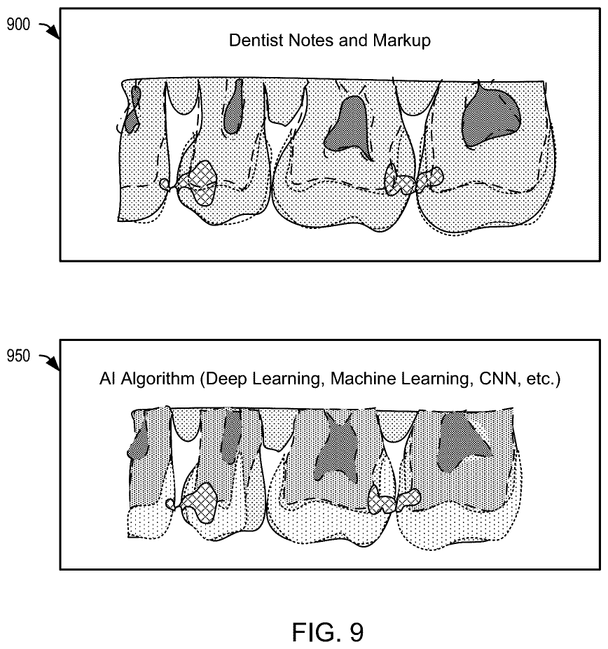

FIG. 9 illustrates images 900 and 950 showcasing the capabilities of artificial intelligence to identify pathologies in X-ray images. The presently described systems and method propose leveraging medical and dental image analysis capabilities to automatically generate narratives for specified dental codes in dental insurance claims (or medical codes in dental and/or medical insurance claims) that are customized based on the specific characteristics of the X-ray as identified by the AI imagine analysis system.

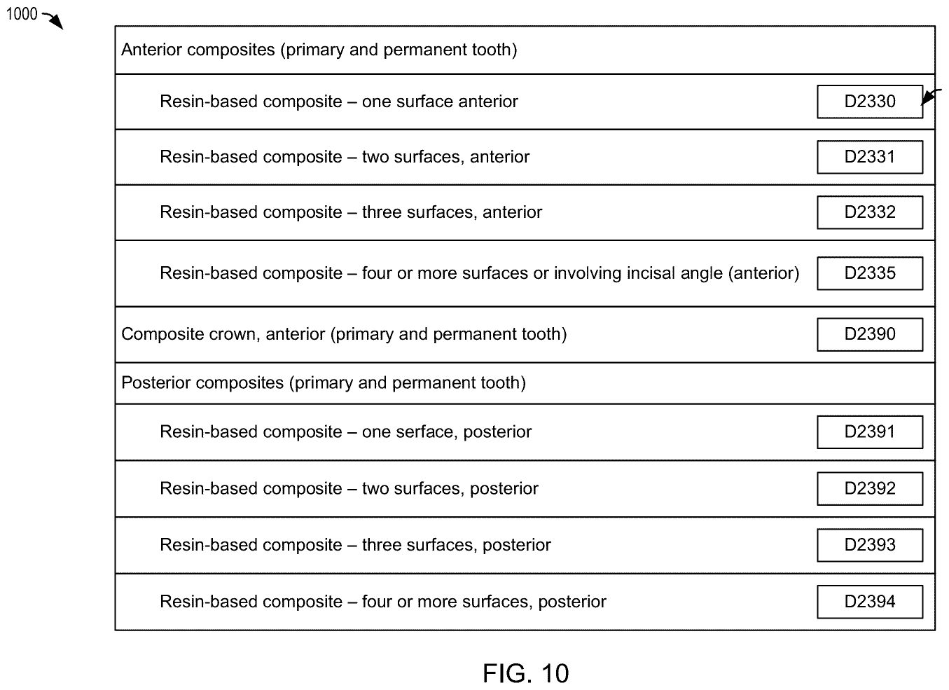

FIG. 10 illustrates a list of dental codes 1000 that might be entered into a claim in connection with work performed on a patient whose X-rays correspond to those illustrated in FIG. 9. The co-pilot portal computing system 325 in FIG. 3A may process a dental claim that includes the codes 1000 illustrated in FIG. 10. The co-pilot portal computing system 325 may retrieve an untagged and unmarked X-ray and submit it to the AI-based medical imaging analysis system 335. The AI-based medical imaging analysis system 335 may return a tagged, marked-up, colored, annotated, metadata-embedded, or otherwise computer-encoded version of the X-ray (similar to that shown on the bottom image in FIG. 9) that identifies various characteristics, pathologies, or other information relevant to one or more of the dental codes shown in FIG. 10.

As an example, the initial claim may include an entry that reads “D2391, Tooth #19, Occlusal Surface.” The co-pilot portal computing system 325 may query a database to determine that the dental code and tooth number correspond to a bitewing X-ray. The co-pilot portal computing system 325 may retrieve (e.g., via an API) the most recent bitewing X-ray from the dental office practice management system 350. The co-pilot portal computing system 325 may transmit the retrieved X-ray to the medical imaging analysis system 335 to search for occlusal decay on tooth number 19. The medical imaging analysis system 335 may utilize various AI-based medical imaging algorithms, such as machine learning models trained for dental image analysis, to identify tooth number 19 and return information characterizing the condition and characteristics of tooth number 19 that are relevant to the D2391 dental code.

The narrative generation module may utilize this information to generate a narrative to be added to the claim. The narrative may utilize various abbreviations and terminology common in the industry, prioritize some types of information over other types of information (e.g., as specified in a set of rules or rankings), and/or maintain the total length within a standard character limitation (e.g., fewer than 75 characters for standard ADA dental claim forms).

For example, without any character limitation, the narrative generation module may utilize the information returned by the medical imaging analysis system 335 to generate a narrative that reads, “Caries present on tooth number 19 occlusal surface through the enamel penetrating approximately 2 mm into dentin requiring a restoration, 20 mm from the port chamber, no bone loss, healthy to structure.” The narrative generation module may shorten the narrative to be within specified character limitations using standardized abbreviations to read “19-O caries 2 mm into dentin requires treatment.”

As another example, the system may receive a claim with a dental code D4341, LR. The system retrieves the most recent bitewing X-ray and submits the X-ray for AI-based image processing to identify bone loss and calculus. Based on the specific information returned from the AI-based image processing, the system may determine (1) 1.5 to 3.1 mm of bone loss: teeth numbers 28, 29, 30, 31; (2) that there is no furcation involvement, and (3) calculus present 31 Mesial, two M three distal M #4 distal. The narrative generation module may generate a narrative for inclusion in the claim in connection with the D4341 dental code that reads “20% bone loss 28, 29, 31. Calculus 31-M. Requires SRP.”

In various embodiments, the system has access to a database that associates each CDT code with (i) a specific type of X-ray (e.g., PA, BW, CBCT, PAN, etc.); (ii) a periodontal charting; and/or (iii) intraoral pictures. In some embodiments, the AI-based image analysis system is configured to analyze the X-rays and generate the appropriate narrative. In other embodiments, the AI-based image analysis system returns information characterizing the X-ray in a format that can be interpreted by a separate AI-based narrative generation system. Thus, whether implemented as a single AI-based system or as multiple stages of AI-based systems or subsystems, the overall system operates to report findings related to the health and disease state of the area of the mouth in a particular X-ray, create a narrative or chart notes, and provide a summary using approved abbreviations starting with the most important information and staying with any specified character ranges, maximums, minimums, or specified limitations.

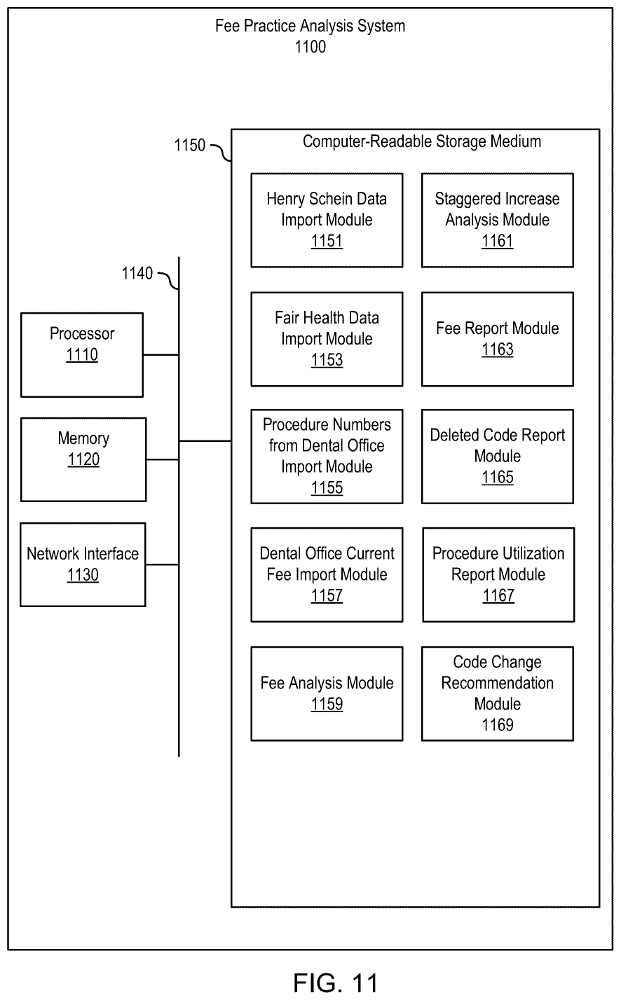

FIG. 11 illustrates a block diagram of a fee practice analysis computing system 1100, according to one embodiment. The presently described system provides a solution to the inefficiencies and manual processes associated with comparing dental procedure fees within a specific geographic region, identifying fee discrepancies, and making adjustments to optimize revenue for dental practices. The process cannot be reasonably implemented manually due to the impossibility of comparing large amounts of data that are well beyond the comprehension of any single human, the impossibility of dynamically updating such data in real time via a graphical user interface in response to target goal selections by a user, and the impossibility of incorporating dynamic feedback based on ongoing insurance carrier acceptance data. The system aims to assist dental offices in maximizing their revenue by ensuring that their fees are competitive and in line with industry standards, while also avoiding potential issues with insurance carriers and/or complaints from individual patients.

As illustrated, the system 1100 includes a bus that connects a processor 1110, memory 1120, network interface 1130, and a computer-readable storage medium 1150. The computer-readable storage medium includes various modules 1151-1169 for importing data, processing the data, and generating a report. For example, a Henry Schein data import module 1151 imports fee data from a Henry Schein dataset known to those of skill in the art. Similarly, the Fair Health data import module 1153 imports fee data from a Fair Health dataset known to those of skill in the art. The procedure numbers from dental office import module 1155 obtains a list of the procedures performed by the dental office and the dental office current fee import module 1157 obtains information identifying the fee charged for each procedure performed.

The data import modules of the system accept input data in the form of procedure codes, medical or dental codes, procedure descriptions, quantities, associated fees, dates of service, payments actually received, adjustments to payments, and the like. The system offers the capability to import data from various file formats, including PDF and Excel, eliminating or reducing the need for manual data entry. The quantity of data may not be manually enterable, such that the presently described systems and methods cannot be reasonably performed by individuals. The sheer volume of data processed and analyzed makes manual execution impractical, underscoring the advanced automation needed for accurate and efficient fee analysis. The quantity of data may not be manually enterable, such that the presently described systems and methods cannot be reasonably performed by individuals.

A fee analysis module 1159 automatically compares the fees of dental procedures with fees charged by other practices within the same geographic area (e.g., zip code) with the data from Henry Schein, Fair Health, and/or other datasets. The fee analysis module 1159, in some embodiments, averages the multiple data sources to provide a more comprehensive and balanced benchmark. The system may use this information to calculate where a dental practice's fees rank in comparison to peers, identifying discrepancies where fees are higher or lower than expected. This nuanced multi-source comparison (e.g., average multi-source analysis) ensures that outlier data is balanced out, resulting in more accurate recommendations for fee adjustments. The system calculates where a dental practice's fees rank in comparison to peers, identifying discrepancies where fees are higher or lower than expected.

The system may include a fee increase analysis recommendation module, such as a staggered increase analysis module 1161 to stagger the recommended fees over several weeks, months, or even years to reduce negative perceptions and/or pushback from payors. The staggered increase analysis module 1161 may provide recommendations for incremental fee adjustments, addressing the risk of sudden fee hikes that might lead to patient pushback or insurance denials. For example, to bridge the gap between current fees and a target percentile level (e.g., 85th percentile), the system can recommend increases at pre-set intervals. For instance, if a target fee for a specific dental code is $100 and the current fee is $60, instead of an immediate $40 increase, the system may propose a staggered plan: an increase of 14% every six months, progressing through intermediary steps (e.g., $68, $78, $88) until reaching the target fee. In various embodiments, this staged approach balances profitability with patient satisfaction and insurance compliance.

The fee report module 1163 may provide a report of the fees and recommendations for immediate fee increases and/or staged or staggered future fee increases. The system may generate comprehensive reports for dental practices, including fee adjustment recommendations, deleted code analysis, patient-based revenue potential, and the like. In some embodiments, the reports may include educational content to guide practices on proper code usage. In some embodiments, the fee report module 1163 may generate a report with a breakdown of adjustments needed, highlights of potential revenue gains, percentile ranking of fees, and educational content to inform dental practices about code usage and compliance. The report ensures that practices are aware of the rationale behind recommendations and prepared for potential patient or insurer inquiries.

A deleted code report module 1165 may identify instances where deleted (e.g., expired) procedure codes have been used and recommend correct codes to maximize reimbursement amounts and/or minimize delayed processing times. This feature helps dental practices avoid the financial pitfalls associated with outdated coding and improves the likelihood that claims are processed efficiently and accurately.

The system may also include a procedure utilization report module 1167 to compare the utilization of specific dental codes and implemented procedures to common practices. For example, the system may analyze patient demographics to identify missed revenue opportunities, such as unclaimed codes for specific dental conditions. If, for example, the industry has identified a typical rate of incidence for specific conditions, (e.g., gingivitis, cavities, etc.), the system may check to see if the dental office's procedures correspond to expected occurrence rates.

The system may also recommend that different fee codes be used (e.g., via a code change recommendation module 1169) if higher fees can be achieved using different codes and/or if recommended fee increases can be achieved in a shorter amount of time using a different code.

The presently described systems and methods improve upon existing technologic tools available to dental practices by providing an automated system for fee comparison and optimization within specific geographic regions, providing recommendations for splitting fee adjustments into two or more stages to mitigate patient/insurer dissatisfaction, and identifying and rectifying the use of deleted procedure codes.

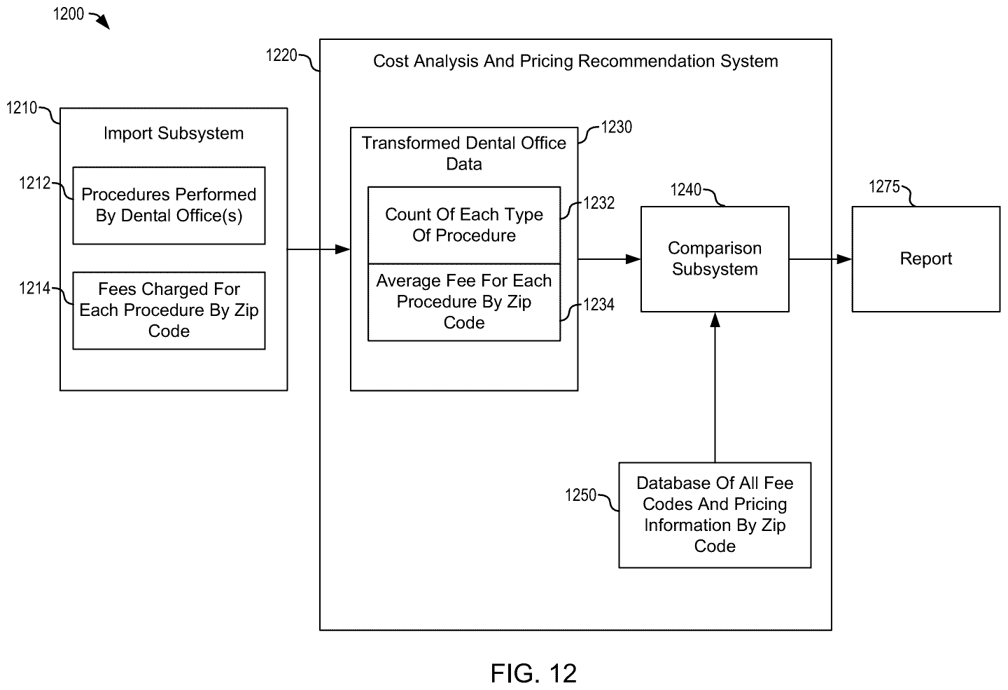

FIG. 12 illustrates a block diagram 1200 of an example cost analysis and pricing recommendation system 1220, according to one embodiment. As illustrated, an import subsystem 1210 may import information identifying the procedures performed by the dental office 1212 and the fees charged for each procedure by zip code (or other geographic region) 1214. The import subsystem 1210 is capable of accepting data in various formats, such as CSV, Excel, and PDF, facilitating seamless integration with existing dental office systems. The information may be processed and/or otherwise transformed, at 1230, into usable data with a count of the number of times each procedure is performed by the dental office 1232 along with the fee charged, paid, and/or adjusted 1234. The system automates the data validation process to ensure that imported data aligns with expected formats and standards, significantly minimizing manual data entry errors.

The system has access to a database 1250 of fee information for all fee codes. However, the system may only compare and report on those fee codes for which the dental office actually performs work in excess of a threshold number of times during a given time period. For example, the system 1220 may report on every fee code performed by the dental office, or only on those fee codes for which the dental office performed at least X procedures during the last Y months, where X and Y are integer values.

A comparison subsystem 1240 compares the information from the dental office with the information from the database of information to generate a report. As previously described, the report 1275 may be based on a comparison of the fees of dental procedures with fees charged by other practices within the same geographic area (e.g., zip code). The report 1275 may identify where a dental practice's fees rank in comparison to peers in terms of percentile or averages. The report 1275 may provide recommendations for fee adjustments to bring a dental practice's fees in line with a target percentile of fees charged by their peers (e.g., same type of practice, geographic area, etc.).

The report 1275 may recommend splitting fee adjustments into two or more stages to avoid abrupt changes and patient dissatisfaction. This phased approach allows for smoother financial transitions, balancing patient expectations with revenue optimization. The report 1275 may also recommend updating the usage of some codes or discontinuing the use of some expired codes. The report 1275 may include an analysis of patient-based revenue potential and/or provide educational material, as described above. For example, the system 1220 may identify that the dental office has X number of adult patients, that 50% of those patients statistically have gingivitis (from data within a database), and that the dental office has only performed gingivitis-related procedures for 10% of its patients. The system 1220 may report that an increase in gingivitis related procedures is probably justified and indicate the amount of revenue available if the expected number of procedures were performed.

The system 1220 may enhance the efficiency of insurance claim processing in the healthcare industry, including in the dental industry. The system 1220 digests office reports in paper format, electronic format, via an integrated application, or an application programming interface (API). Additionally, the system 1220 can flag inconsistencies between the dental office's data and external insurance or regulatory standards, allowing practices to correct issues proactively. The system cross-references the data from the dental office with various data sources to improve the accuracy and success of insurance claims.

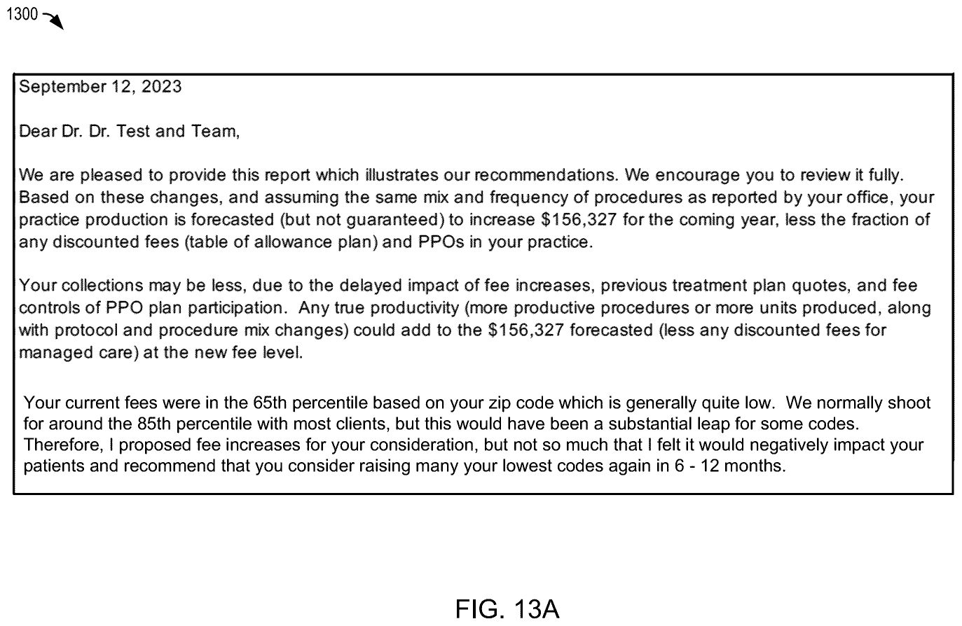

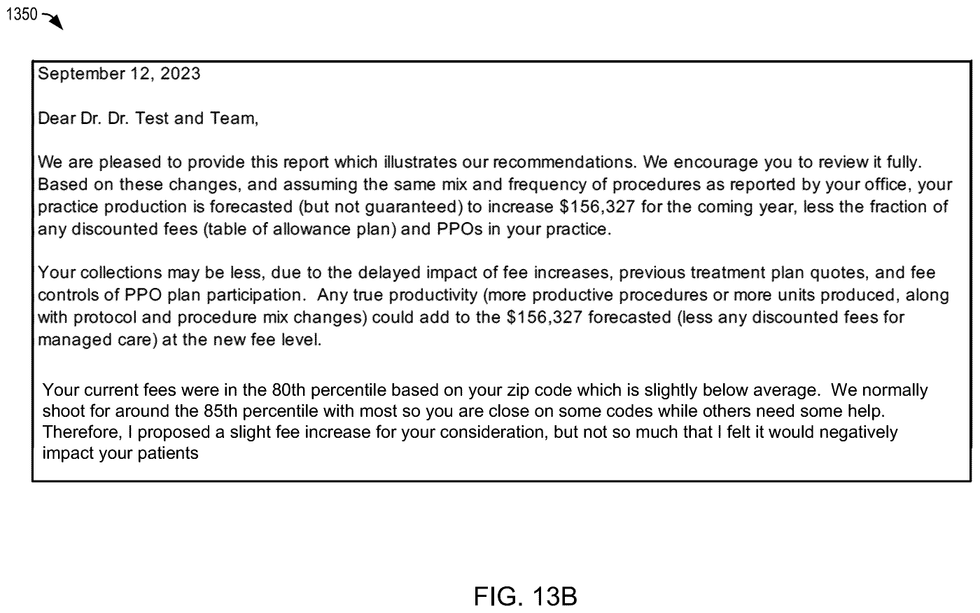

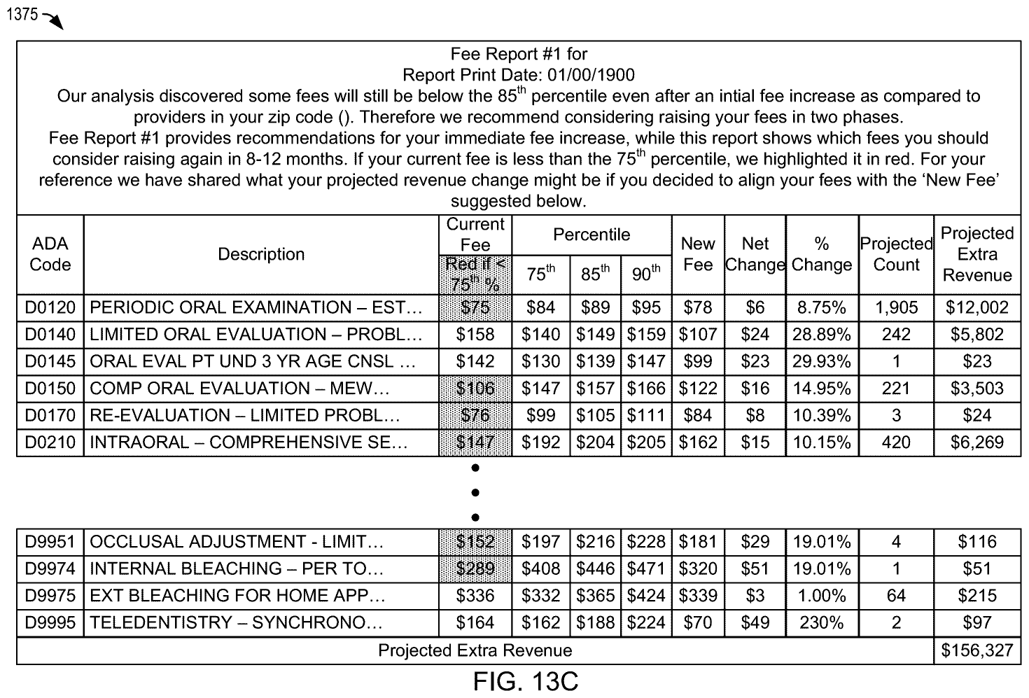

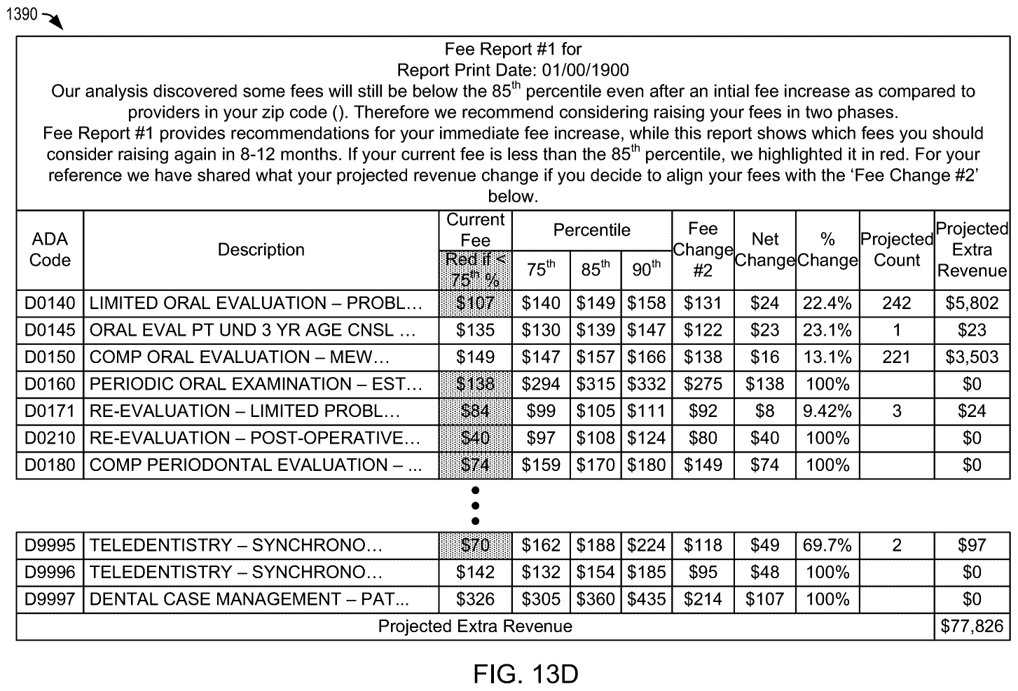

FIG. 13A illustrates an example of a first portion 1300 of a fee practice report with staggered or staged fee increase recommendations, according to one embodiment. The system may automatically generate the report and paragraphs based on the specific data. The illustrated example is a header of a report for a dental office whose fees are in the 65th percentile.

FIG. 13B illustrates an example of a first portion 1350 of a fee practice report with staggered or staged fee increase recommendations, according to one embodiment. The system may automatically generate the report and paragraphs based on the specific data. The illustrated example is a header of a report for a dental office whose fees are in the 80th percentile.

FIG. 13C illustrates an example fee report 1375 of an immediate fee increase plan, according to one embodiment. As illustrated, the report 1375 includes a table that can be sorted by any column to allow for quick analysis of fee data. The table includes fee codes or dental codes (e.g., ADA codes), a description of each code, the current fee (highlighted if it falls below an established percentile threshold), target fees for various percentiles for the geographic region, proposed new fees, the fee increase as a percentage of the current fee, the projected number of procedures to be performed in the coming year (or month or other time period), and the projected revenue increase for the relevant time period.

The report 1375 also includes additional data validation flags to indicate if any anomalies or errors were detected during the data import or analysis process. For example, certain columns may highlight deleted codes, custom codes, or codes with data entry discrepancies, alerting the dental office to potential compliance or accuracy issues. The inclusion of such checks ensures that practices avoid using outdated or non-standard codes, thereby reducing the likelihood of insurance denials and enhancing claim acceptance rates.

A notable feature of the report is its customization capability. The system can simulate various fee increase scenarios based on provider-specified targets. Thus, the report may be presented in the form of a dynamically updated graphical user interface. Any adjustment to a parameter causes the depend data to be automatically updated. For example, the report 1375 may include an adjustable parameter for the maximum allowable fee increase percentage to avoid triggering insurance company alerts or patient dissatisfaction. The system may present phased increase options, showing how incremental adjustments would impact revenue over different periods, such as immediate changes followed by secondary increases after six to eight months. These stages help practices align their pricing strategy with patient expectations and insurance policies, ensuring smoother transitions and revenue sustainability.

The report may include conditional logic-driven notes explaining the rationale behind each recommended adjustment. For example, if a code's proposed increase exceeds a predetermined limit, a note may explain the reasoning, referencing typical insurance company thresholds or industry standards. Additionally, the system provides automated override options, allowing providers to apply specific fee limits or adjustments if certain conditions or provider preferences are met.

The fee report 1375 may also provide a comprehensive analysis of underutilized codes, revealing areas where the practice may not be fully leveraging its billing potential. In some embodiments, the report 1375 cross-references patient demographics, indicating when a higher number of patients could justify increased utilization of certain codes, such as periodontal treatments or diagnostic procedures, and how those changes could influence overall revenue.

FIG. 13D illustrates an example fee report 1390 for future fee increases by dental billing code, according to one embodiment. The same table headers are used, and the data may be sorted and/or filtered by any column (not illustrated). The report 1390 outlines a multi-stage fee increase plan aimed at gradually aligning a dental practice's fees with a target percentile of peers in their geographic area. While the illustrated example includes only a 2-stage fee increase, it is appreciated that any number of stages may be used to achieve optimal results. The system can be configured to support phased fee adjustments over an extended period, such as three, four, or even five stages, allowing for more granular and controlled fee adjustments.

In some embodiments, the report 1390 may include a breakdown of projected revenue increases for each stage, providing clear visibility into the financial impact of incremental changes. For instance, stage one may reflect an initial 10% increase in fees, while stage two raises fees further to meet the 85th percentile target. The flexibility to customize the stages based on the provider's comfort level, insurance carrier regulations, and competitive landscape ensures that dental practices can align their pricing strategy effectively.

In some embodiments, the report 1390 highlights codes that are currently underutilized or overutilized. This analysis may be integrated with future fee recommendations. As such, the report 1390 may guide a practice on how to adjust the fees and the frequency and correctness of code usage. For example, the report may flag codes that are rarely used but could represent a significant revenue opportunity if utilized more frequently. Conversely, the report 1390 may also identify codes that are overused that may trigger audits or denials from insurance carriers.

In various embodiments, the dental office can set maximum allowable fees for certain codes, ensuring that they remain competitive without surpassing patient or insurance expectations. These overrides, combined with dynamic feedback and customization options, provide dental practices with the control needed to balance their financial goals with patient affordability and regulatory compliance.

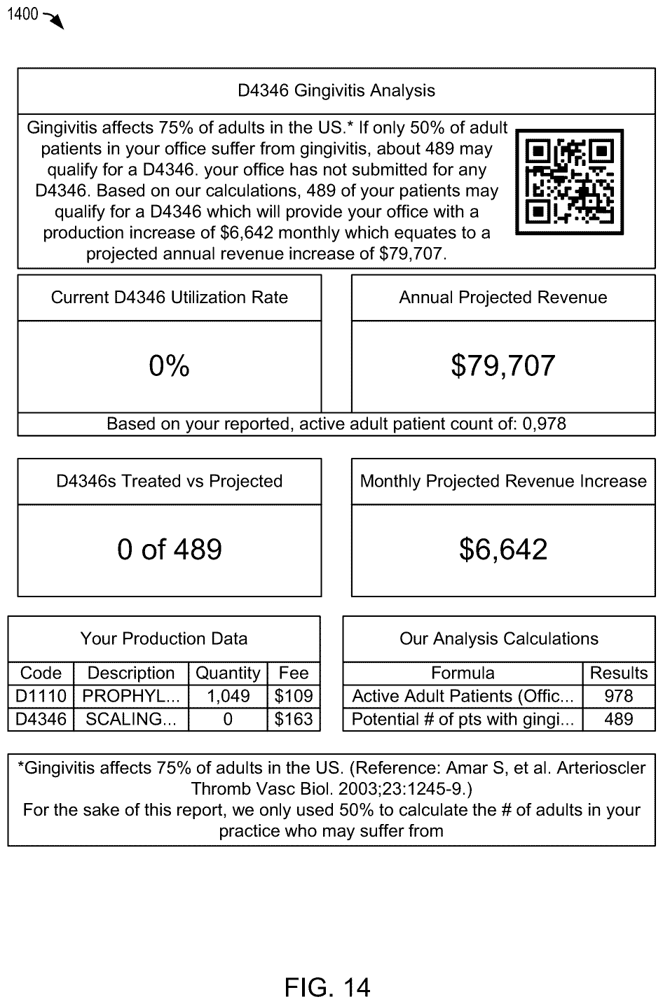

FIG. 14 illustrates an example utilization report 1400 for an example dental code with a projected increase in revenue, according to one embodiment. As illustrated, the report 1400 summarizes a description of the dental code, current practice utilization, and projected revenue with expected utilization. As illustrated, the report 1400 summarizes a 0% utilization rate and indicates that since 75% of adults have gingivitis, at least 50% of the dental office's patients (489 patients) are likely to need care that would result in an increase in monthly revenue of $6,642.

Some of the infrastructure that can be used with embodiments disclosed herein is already available, such as general-purpose computers, computer programming tools and techniques, digital storage media, and communications networks. A computer may include a processor, such as a microprocessor, microcontroller, logic circuitry, or the like. The processor may include a special-purpose processing device, such as an ASIC, a PAL, a PLA, a PLD, a CPLD, a Field Programmable Gate Array (FPGA), or another customized or programmable device. The computer may also include a computer-readable storage device, such as non-volatile memory, static RAM, dynamic RAM, ROM, CD-ROM, disk, tape, magnetic, optical, flash memory, or another computer-readable storage medium.

Suitable networks for configuration and/or use, as described herein, include any of a wide variety of network infrastructures. Specifically, a network may incorporate landlines, wireless communication, optical connections, various modulators, demodulators, small form-factor pluggable (SFP) transceivers, routers, hubs, switches, and/or other networking equipment.

The network may include communications or networking software and may operate using TCP/IP, SPX, IPX, SONET, and other protocols over twisted pair, coaxial, or optical fiber cables; telephone lines; satellites; microwave relays; modulated AC power lines; physical media transfer; wireless radio links; and/or other data transmission “wires.” The network may encompass smaller networks and/or be connectable to other networks through a gateway or similar mechanism.

Aspects of certain embodiments described herein may be implemented as software modules or components. As used herein, a software module or component may include any type of computer instruction or computer-executable code located within or on a computer-readable storage medium, such as a non-transitory computer-readable medium. A software module may, for instance, comprise one or more physical or logical blocks of computer instructions, which may be organized as a routine, program, object, component, data structure, etc., that perform one or more tasks or implement particular data types, algorithms, and/or methods.

A particular software module may comprise disparate instructions stored in different locations of a computer-readable storage medium, which together implement the described functionality of the module. Indeed, a module may comprise a single instruction or many instructions and may be distributed over several different code segments, among different programs, and across several computer-readable storage media. Some embodiments may be practiced in a distributed computing environment where tasks are performed by a remote processing device linked through a communications network. In a distributed computing environment, software modules may be located in local and/or remote computer-readable storage media. In addition, data being tied or rendered together in a database record may be resident in the same computer-readable storage medium or across several computer-readable storage media and may be linked together in fields of a record in a database across a network.

The various functional components of the described systems and methods may be modeled as a functional block diagram that includes one or more remote terminals, networks, servers, data exchanges, and software/hardware/firmware modules configured to implement the various functions, features, methods, and concepts described herein. In many instances, each application, embodiment, variation, option, service, and/or another component of the systems and methods described herein may be implemented as a module of a larger system. Each module may be implemented as hardware, software, and/or firmware, as would be understood by one of skill in the art for the particular functionality, and may be part of a larger physical system that may include computer-readable instructions, processors, servers, endpoint computers, and/or the like.

The embodiments of the disclosure can be understood by reference to the drawings, wherein like parts are designated by like numerals throughout. The components of the disclosed embodiments, as generally described and illustrated in the figures herein, could be arranged and designed in a wide variety of different configurations. Further, those of skill in the art will recognize that one or more of the specific details may be omitted, or other methods, components, or materials may be used. In some cases, operations are not shown or described in detail. Thus, the detailed description of the embodiments of the systems and methods of the disclosure is not intended to limit the scope of the disclosure, as claimed, but is merely representative of possible embodiments.

What is claimed is:

1. A system for generating customized fee schedules for a dental practice, comprising:
a processor configured to execute instructions;
a memory operatively connected to the processor; and
a computer-readable medium storing instructions that, when executed by the processor, cause the system to:
receive input parameters, including:
(i) a global percentile target,
(ii) a maximum percentage increase per procedure code, and
(iii) a specified number of fee adjustment stages;
retrieve current fee data for multiple procedure codes from a dental practice management system;
analyze the retrieved current fee data and compare it to benchmark fee data aggregated from external sources to determine percentile rankings for each procedure code;
generate a staged fee increase plan for each procedure code, ensuring that:
(i) no single fee increase exceeds the specified maximum percentage,
(ii) adjustments are distributed across the specified number of fee adjustment stages, and
(iii) procedure codes already at or above the percentile target are adjusted by not more than a predefined percentage threshold;
generate a report that includes the customized fee schedule per procedure code, wherein the report includes a timeline for each stage of fee adjustments and projected revenue outcomes for each stage of the fee adjustments based on the fee schedule; and
adjust the report based on user feedback that specifies at least one of: a new global percentile target and a manual override value for at least one procedure code.

2. The system of claim 1, wherein the system applies different percentage increase maximums for codes based on predefined rules for patient sensitivity to price changes.

3. The system of claim 1, further comprising logic to override the external fee data for specific procedure codes and set a fee to a fixed amount based on user-specified criteria.

4. The system of claim 1, wherein the system identifies procedure codes that do not meet the percentile target and outputs a suggested course of action, including phased fee adjustments to reach the percentile target over multiple stages.

5. A dental practice management system for analyzing and structuring procedure fee increases, comprising:
a data import module configured to retrieve multiple fee data sets from independent sources for a specified geographic location;
a data normalization module to normalize the fee data sets;
a fee analysis module that compares a practice's existing fees to percentile-based target fees derived from the normalized data sets and generates recommendations to adjust the fees toward a target percentile;
an error detection module that identifies inconsistencies within imported data and alerts users to correct missing data for accuracy in analysis; and
a reporting module that presents adjusted fees, expected revenue impact, and target percentile positioning for user approval or modification.

6. The system of claim 5, wherein the error detection module validates data by comparing imported fees and procedure counts with historical data stored in a database to identify inaccuracies.

7. The system of claim 5, wherein the fee analysis module provides a breakdown of revenue potential for each fee adjustment based on current and projected patient visit frequency.

8. The system of claim 5, wherein the error detection module identifies missing demographic data that are necessary for accurate revenue predictions.

9. The system of claim 5, further comprising a percentile-based fee adjustment module that allows adjustments to fees based on percentile target thresholds selected by a practitioner.

10. The system of claim 5, wherein the reporting module displays revenue changes based on selected percentile goals across one or more stages.

11. The system of claim 5, further comprising a patient feedback analysis module that calculates optimal fee adjustments based on historical acceptance rates and insurance provider feedback trends.

12. The system of claim 5, wherein the fee analysis module recommends fee stages over a user-defined timeframe to gradually increase fees and minimize patient dissatisfaction.

13. The system of claim 5, wherein the data import module applies data normalization techniques to adjust for fee variations across multiple sources and formats.

14. The system of claim 5, further comprising an override feature allowing users to manually adjust individual fees despite overall percentile-based recommendations.

15. A method for generating a structured report for dental practice fee adjustment based on utilization data, the method comprising:
retrieving procedural frequency and fee data from a dental practice management system;
integrating the retrieved data with external sources of fee information to calculate a baseline percentile for each procedure fee relative to local standards;
determining an adjustment path that recommends incremental fee increases, frequency of increase, and final target percentile positioning, based on historical patient feedback and insurance company acceptance rates; and
providing a dynamic report interface that allows practitioners to:
select a staging frequency for the incremental fee increases,
adjust the final target percentile positioning,
visualize potential revenue impacts of the selected staging frequency and final target percentile positing.

16. The method of claim 15, further comprising setting a maximum increase limit for fee adjustments based on insurance company feedback and practice revenue goals.

17. The method of claim 15, further comprising dynamically updating the fee structure by adjusting fee recommendations based on cumulative insurance feedback and underutilization rates.

18. A computerized method for improving compliance and optimizing revenue from insurance claims in dental practices, comprising:
identifying and removing deprecated and custom dental procedure codes from a practice's records and replacing them with standard codes;
calculating underutilized codes based on patient demographics and providing utilization recommendations to capture previously unbilled procedures;
using historical data on insurance denial and adjustment trends to provide tailored guidance on maximizing reimbursements by applying correct procedure codes and recommended narratives; and
dynamically updating a predictive model based on continuous data entry from insurance feedback to improve future coding and billing accuracy.

19. The method of claim 18, further comprising automatically generating narratives for commonly performed procedures based on pathology data from medical imaging and historical claim feedback.

20. The method of claim 18, further comprising dynamically adjusting recommended codes and narratives based on past successful appeals and denied claims.

21. The method of claim 18, further comprising tracking frequently underutilized procedure codes and recommending billing adjustments based on patient demographics and standard treatment protocols.
